(19) United States
(12) Patent Application Publication
(10) Pub. No.: US 2010/0331646 A1
(43) Pub. Date: Dec. 30, 2010

(54) Health Management Devices and Methods

(75) Inventors: Udo Hoss, Castro Valley, CA (US); Benjamin Jay Feldman, Oakland, CA (US); Hyun Cho, Berkeley, CA (US); Marc Barry Taub, Mountain View, CA (US); Gary Ashley Stafford, Hayward, CA (US)

Correspondence Address: JACKSON & CO., LLP, 6114 LA SALLE AVENUE, #507, OAKLAND, CA, 94611-2802, US

(73) Assignee: Abbott Diabetes Care Inc., Alameda, CA (US)

(21) Appl. No.: 12/495,795

(22) Filed: Jun. 30, 2009

(51) Int. Cl. A61B 5/1468

(52) U.S. Cl. 600/347

(57) ABSTRACT

Methods and devices to detect analyte in body fluid are provided. Embodiments include analyte sensors designed so that at least a portion of the sensor is positionable beneath a skin surface during analyte monitoring.

### BACKGROUND

The detection of the level of analytes, such as glucose, lactate, oxygen, and the like, in certain individuals is vitally important to their health. For example, the monitoring of glucose is particularly important to individuals with diabetes. Diabetics may need to monitor glucose levels to determine when insulin is needed to reduce glucose levels in their bodies or when additional glucose is needed to raise the level of glucose in their bodies.

There are many instances in which a testing component is positioned beneath a skin surface of a user. It may be desirable, if not necessary, to be able to select a depth of positioning. For example, U.S. Pat. No. 6,175,752 describes in vivo monitoring systems to monitor an analyte such as glucose. Embodiments include in vivo analyte sensors that are positioned beneath a skin surface of a user, where they may reside for a period of time to monitor one or more analytes in biological fluid contacted with the analyte sensor.

Accordingly, of interest are medical device systems such as analyte monitoring systems that are able to be selectively positioned at a given depth beneath the skin, which depth is selected from a plurality of depths.

### SUMMARY

Embodiments include medical device systems and methods that include a selective depth controller to select a depth to position at least a portion of a medical device system beneath a skin surface of a user. Embodiments of the subject invention may be applicable to a variety of medical devices. However, embodiments herein are described primarily with respect to in vivo, transcutaneously positioned analyte monitoring systems, e.g., glucose monitoring systems, for exemplary purposes only. Such descriptions are in no way intended to limit the scope of the subject invention.

Embodiments include in vivo glucose monitoring systems that include one or more depth controllers that may be set to a selected depth prior to positioning at least a portion of an analyte sensor of the system in a user. Embodiments include transcutaneous analyte systems that include transcutaneous analyte sensors that are configured to be positioned in a user to a plurality of settable, predetermined depths, and provide a depth indicator so a user may pre-set the system to a given depth. For example, provided are transcutaneous analyte systems in which the depth of the analyte sensor in tissue is adjustable.

Certain embodiments include an analyte sensor mateable, e.g., electrically and/or physically, etc., with a second component that is an on-body unit, e.g., configured to be attached to a skin surface, such as a sensor control unit and/or a mounting unit that may be used, e.g., to mount a sensor control unit or the like. In certain embodiments, the distance between the sensor and the on-body unit is selectively variable, i.e., adjustable. In certain embodiments, the distance between the sensor and the on-body unit is fixed, i.e., this distance is not adjustable so that the sensor is configured to always extend the same distance from the on-body unit each time it is used, regardless of the selected depth.

### BRIEF DESCRIPTION OF THE DRAWINGS

FIG. 1 shows a block diagram of an embodiment of a data monitoring and management system according to the present invention;

FIG. 2 shows a block diagram of an embodiment of the transmitter unit of the data monitoring and management system of FIG. 1;

FIG. 3 shows a block diagram of an embodiment of the receiver/monitor unit of the data monitoring and management system of FIG. 1;

FIG. 4 shows a schematic diagram of an embodiment of an analyte sensor according to the present invention;

FIGS. 5A-5B show a perspective view and a cross sectional view, respectively of another embodiment of an analyte sensor;

FIG. 6 shows an embodiment of a depth controllable analyte system that includes a selective depth controller that includes an offset;

FIG. 7 shows an embodiment of a depth controllable analyte system that includes a selective depth controller that includes an offset of a plurality of depth adjust spacers;

FIG. 8 shows an embodiment of a depth controllable analyte system that includes a selective depth controller integrated in the housing of an on-body unit;

FIGS. 9A and 9B show embodiments of depth controllable analyte systems that include a selective depth controller that selectively controls the angle of sensor insertion;

FIGS. 10A and 10B show an embodiment of a depth controllable analyte system that includes a selective depth controller that includes a bladder, wherein FIG. 10A shows the bladder in a deflated state and FIG. 10B shows the bladder in an inflated state to position the sensor at a selected depth;

FIG. 11 shows an embodiment of a mounting unit that includes a selective depth controller integrated therewith, and which is configured to receive a sensor control unit and associate with an analyte sensor;

FIG. 12 shows an embodiment of a mounting unit mated with a sensor control unit that includes a selective depth controller integrated therewith; and

FIGS. 13A-13G an embodiment of a depth controllable analyte system that includes an inflatable cuff to selectively adjust the depth of insertion of a sensor.

### DETAILED DESCRIPTION

Before the present invention is described, it is to be understood that this invention is not limited to particular embodiments described, as such may, of course, vary. It is also to be understood that the terminology used herein is for the purpose of describing particular embodiments only, and is not intended to be limiting, since the scope of the present invention will be limited only by the appended claims.

Where a range of values is provided, it is understood that each intervening value, to the tenth of the unit of the lower limit unless the context clearly dictates otherwise, between the upper and lower limit of that range and any other stated or intervening value in that stated range, is encompassed within the invention. The upper and lower limits of these smaller ranges may independently be included in the smaller ranges is also encompassed within the invention, subject to any specifically excluded limit in the stated range. Where the stated range includes one or both of the limits, ranges excluding either or both of those included limits are also included in the invention.

It must be noted that as used herein and in the appended claims, the singular forms “a”, “an”, and “the” include plural referents unless the context clearly dictates otherwise.

As will be apparent to those of skill in the art upon reading this disclosure, each of the individual embodiments described and illustrated herein has discrete components and features which may be readily separated from or combined with the features of any of the other several embodiments without departing from the scope or spirit of the present invention.

The figures shown herein are not necessarily drawn to scale, with some components and features being exaggerated for clarity.

Generally, embodiments of the present invention relate to methods and devices for detecting at least one analyte such as glucose in body fluid. Embodiments relate to the continuous and/or automatic in vivo monitoring of the level of one or more analytes using a continuous analyte monitoring system that includes an analyte sensor at least a portion of which is to be positioned beneath a skin surface of a user for a period of time and/or the discrete monitoring of one or more analytes using an in vitro blood glucose (“BG”) meter and an analyte test strip. Embodiments include combined or combinable devices, systems. kits and methods and/or transferring data between an in vivo continuous system and a BG meter system.

Accordingly, embodiments include analyte monitoring devices and systems that include an analyte sensor—at least a portion of which is positionable beneath the skin of the user—for the in vivo detection, of an analyte, such as glucose, lactate, oxygen, and the like, in a body fluid. Embodiments include wholly implantable analyte sensors and analyte sensors in which only a portion of the sensor is positioned under the skin and a portion of the sensor resides above the skin, e.g., for contact to a control unit, mounting unit, transmitter, receiver, transceiver, processor, etc., and combinations thereof. The sensor may be, for example, configured to be subcutaneously positionable in a patient for the continuous or periodic monitoring of a level of an analyte in a patient's interstitial fluid. For the purposes of this description, continuous monitoring and periodic monitoring will be used interchangeably, unless noted otherwise. The sensor response may be correlated and/or converted to analyte levels in blood or other fluids. In certain embodiments, an analyte sensor may be positioned in contact with interstitial fluid to detect the level of glucose, which detected glucose may be used to infer the glucose level in the patient's bloodstream. Analyte sensors may be insertable into a vein, artery, or other portion of the body containing fluid. Embodiments of the analyte sensors of the subject invention may be configured for monitoring the level of the analyte over a time period which may range from minutes, hours, days, weeks, a month or months, or longer.

Of interest are analyte sensors, such as glucose sensors, that are capable of in vivo detection of an analyte for about one hour or more, e.g., about a few hours or more, e.g., about a few days of more, e.g., about three or more days, e.g., about five days or more, e.g., about seven days or more, e.g., about several weeks or at least one month, e.g., multiple months. Future analyte levels may be predicted based on information obtained, e.g., the current analyte level at time to, the rate of change of the analyte, etc. Predictive alarms may notify the user of a predicted or anticipated analyte levels that may be of concern in advance of the user's analyte level reaching the future level. This provides the user an opportunity to take corrective action in a timely manner.

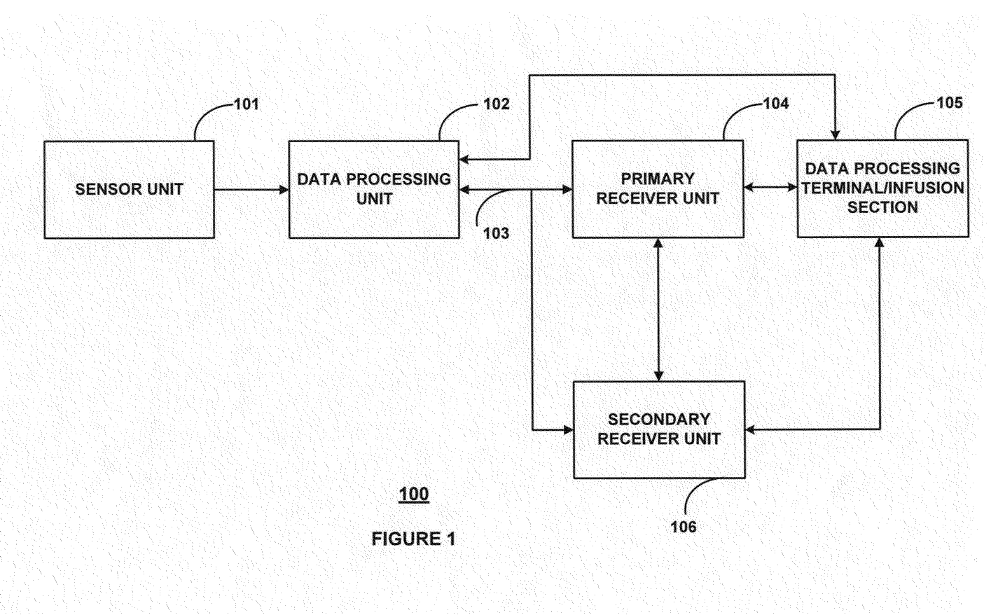

FIG. 1 shows a data monitoring and management system such as, for example, an analyte (e.g., glucose) monitoring system 100 in accordance with certain embodiments. Embodiments of the subject invention are further described primarily with respect to glucose monitoring devices and systems, and methods of glucose detection, for convenience only and such description is in no way intended to limit the scope of the invention. It is to be understood that the analyte monitoring system may be configured to monitor a variety of analytes at the same time or at different times.

Analytes that may be monitored include, but are not limited to, acetyl choline, amylase, bilirubin, cholesterol, chorionic gonadotropin, creatine kinase (e.g., CK-MB), creatine, creatinine, DNA, fructosamine, glucose, glutamine, growth hormones, hormones, ketone bodies, lactate, oxygen, peroxide, prostate-specific antigen, prothrombin, RNA, thyroid stimulating hormone, and troponin. The concentration of drugs, such as, for example, antibiotics (e.g., gentamicin, vancomycin, and the like), digitoxin, digoxin, drugs of abuse, theophylline, and warfarin, may also be monitored. In those embodiments that monitor more than one analyte, the analytes may be monitored at the same or different times.

The analyte monitoring system 100 includes a sensor 101, a data processing unit 102 connectable to the sensor 101, and a primary receiver unit 104 which is configured to communicate with the data processing unit 102 via a communication link 103. In certain embodiments, the primary receiver unit 104 may be further configured to transmit data to a data processing terminal 105 to evaluate or otherwise process or format data received by the primary receiver unit 104. The data processing terminal 105 may be configured to receive data directly from the data processing unit 102 via a communication link which may optionally be configured for bi-directional communication. Further, the data processing unit 102 may include a transmitter or a transceiver to transmit and/or receive data to and/or from the primary receiver unit 104 and/or the data processing terminal 105 and/or optionally the secondary receiver unit 106.

As shown in FIG. 1 the optional secondary receiver unit 106 may be operatively coupled to the communication link and configured to receive data transmitted from the data processing unit 102. The secondary receiver unit 106 may be configured to communicate with the primary receiver unit 104, as well as the data processing terminal 105. The secondary receiver unit 106 may be configured for bi-directional wireless communication with each of the primary receiver unit 104 and the data processing terminal 105. As discussed in further detail below, in certain embodiments the secondary receiver unit 106 may be a de-featured receiver as compared to the primary receiver, i.e., the secondary receiver may include a limited or minimal number of functions and features as compared with the primary receiver unit 104. As such, the secondary receiver unit 106 may include a smaller (in one or more, including all, dimensions), compact housing or embodied in a device such as a wrist watch, arm band, etc., for example. Alternatively, the secondary receiver unit 106 may be configured with the same or substantially similar functions and features as the primary receiver unit 104. The secondary receiver unit 106 may include a docking portion to be mated with a docking cradle unit for placement by, e.g., the bedside for night time monitoring, and/or a bi-directional communication device. A docking cradle may recharge a powers supply.

Only one sensor 101, data processing unit 102 and data processing terminal 105 are shown in the embodiment of the analyte monitoring system 100 illustrated in FIG. 1. However, it will be appreciated by one of ordinary skill in the art that the analyte monitoring system 100 may include more than one sensor 101 and/or more than one data processing unit 102, and/or more than one data processing terminal 105. Multiple sensors may be positioned in a patient for analyte monitoring at the same or different times. In certain embodiments, analyte information obtained by a first positioned sensor may be employed as a comparison to analyte information obtained by a second sensor. This may be useful to confirm or validate analyte information obtained from one or both of the sensors. Such redundancy may be useful if analyte information is contemplated in critical therapy-related decisions. In certain embodiments, a first sensor may be used to calibrate a second sensor.

The analyte monitoring system 100 may be a continuous monitoring system, or semi-continuous, or a discrete monitoring system, where for example, analyte level of a patient or a user may be continuously monitored, and processed for display or output to the user or the patient in a continuous, semi-continuous or discrete mater (for example, upon request from the patient or the user). That is, the monitored analyte level from the sensor in aspects of the present disclosure may be processed for output to the user or the patient on a substantially real time base corresponding to the each sampled analyte level from the sensor. Alternatively, in aspects of the present disclosure, the output or display of the processed analyte level may be provided to the user or the patient on a preset schedule (for example, every five minutes, every ten minutes, every 15 minutes, or the like). In a further aspect, the sampled analyte level received from the sensor may be processed and stored, and provided to the user upon request from the user—for example, based on user demand or in response to a command received from the user or the patient.

In a multi-component environment, each component may be configured to be uniquely identified by one or more of the other components in the system so that communication conflict may be readily resolved between the various components within the analyte monitoring system 100. For example, unique IDs, communication channels, and the like, may be used. Detailed description of the analyte monitoring system including unique IDs may be found in U.S. Pat. No. 6,560,471 and in pending U.S. patent application Ser. No. 11/060,365 entitled “Method and System for Providing Data Communication in Continuous Glucose Monitoring and Management System, each assigned to the assignee of the present application, and the disclosure of each of which are incorporated herein by reference for all purposes.

In certain embodiments, the sensor 101 is physically positioned in or on the body of a user whose analyte level is being monitored. The sensor 101 may be configured to at least periodically sample the analyte level of the user and convert the sampled analyte level into a corresponding signal for transmission by the data processing unit 102. The data processing unit 102 is coupleable to the sensor 101 so that either devices are positioned in or on the user's body, with at least a portion of the analyte sensor 101 positioned transcutaneously. The data processing unit may include a fixation element such as adhesive, belt, strap, or the like, to secure it to the user's body. A mount or base attachable to the user and mateable with the data processing unit 102 may be used. For example, a mount may include an adhesive surface for skin attachment and/or the data processing unit 102 may include an adhesive surface. The data processing unit 102 performs data processing functions, where such functions may include but are not limited to, filtering and encoding of data corresponding to sampled analyte level of the user, for transmission to the primary receiver unit 104 via the communication link 103. In one embodiment, the sensor 101 or the data processing unit 102 or a combined sensor/data processing unit may be wholly implantable under the skin layer of the user.

In certain embodiments, the primary receiver unit 104 may include an RF receiver and an antenna that is configured to communicate with the data processing unit 102 via the communication link 103, and a data processing section for processing the received data from the data processing unit 102 such as data decoding, error detection and correction, data clock generation, data bit recovery, etc., or any combination thereof.

In operation, the primary receiver unit 104 in certain embodiments is configured to synchronize with the data processing unit 102 to uniquely identify the data processing unit 102, based on, for example, an identification information of the data processing unit 102, and thereafter, to periodically receive signals transmitted from the data processing unit 102 associated with the monitored analyte levels detected by the sensor 101.

Referring again to FIG. 1, the data processing terminal 105 may include a personal computer, a portable computer such as a laptop or a handheld device (e.g., personal digital assistants (PDAs), telephone such as a cellular phone (e.g., a multimedia and Internet-enabled mobile phone such as an iPhone, a Blackberry® device, a Palm based mobile communication device, or similar phone), mp3 player, pager, a global positioning system (GPS) device, and the like), drug delivery device, each of which may be configured for data communication with the receiver via a wired or a wireless connection. Additionally, the data processing terminal 105 may further be connected to a data network (not shown) for storing, retrieving, updating, and/or analyzing data corresponding to the detected analyte level of the user.

The data processing terminal 105 may include an infusion device such as an insulin infusion pump (external, ambulatory, implantable, on-body, for example) or the like, which may be configured to administer insulin to patients, and which may be configured to communicate with the primary receiver unit 104 for receiving, among others, the measured analyte level. Alternatively, the primary receiver unit 104 may be configured to integrate an infusion device therein so that the primary receiver unit 104 is configured to administer insulin (or other appropriate drug) therapy to patients, for example, for administering and modifying basal profiles, as well as for determining appropriate boluses for administration based on, among others, the detected analyte levels received from the data processing unit 102. An infusion device may be an external device or an internal device (wholly implantable in a user).

In certain embodiments, the data processing terminal 105, which may include an insulin pump, may be configured to receive the analyte signals from the data processing unit 102, and thus, incorporate the functions of the primary receiver unit 104 including data processing for managing the patient's insulin therapy and analyte monitoring. In certain embodiments, the communication link 103 as well as one or more of the other communication interfaces shown in FIG. 1, may use one or more of: an RF communication protocol, an infrared communication protocol, a Bluetooth enabled communication protocol, an 802.11x wireless communication protocol, or an equivalent wireless communication protocol which would allow secure, wireless communication of several units (for example, per HIPPA requirements), while avoiding potential data collision and interference.

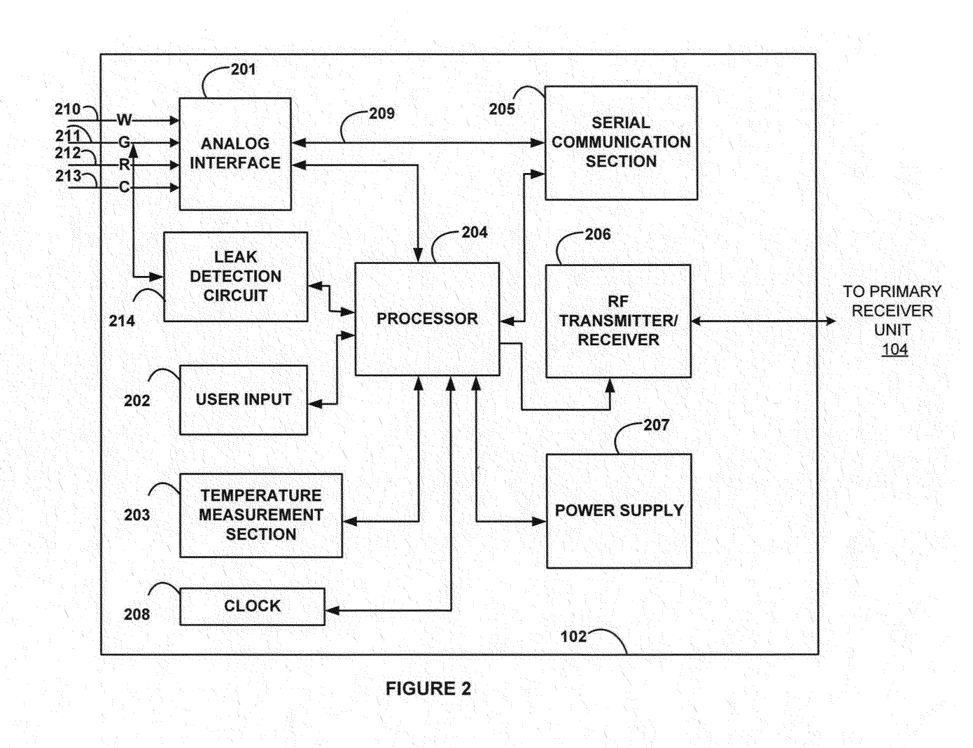

FIG. 2 shows a block diagram of an embodiment of a data processing unit of the data monitoring and detection system shown in FIG. 1. Data processing unit 102 (also referred to as a sensor control unit or a transmitter unit), is configured to receive at least a portion of an analyte sensor, and it, and/or another on-body unit, includes electric contacts for coupling to contacts of the sensor. A sensor control unit may also include a variety of optional components, such as, for example, adhesive for adhering to the skin, a mounting unit, a receiver, a processing circuit, a power supply (e.g., a battery), an alarm system, a data storage unit, a watchdog circuit, and a measurement circuit. These and other optional components are described below. User input and/or interface components may be included or a data processing unit may be free of user input and/or interface components. In certain embodiments, one or more application-specific integrated circuits (ASIC) may be used to implement one or more functions or routines associated with the operations of the data processing unit (and/or receiver unit) using for example one or more state machines and buffers.

As can be seen in the embodiment of FIG. 2, the sensor unit 101 (FIG. 1) includes four contacts, three of which are electrodes—work electrode (W) 210, reference electrode (R) 212, and counter electrode (C) 213, each operatively coupled to the analog interface 201 of the data processing unit 102. This embodiment also shows optional guard contact (G) 211. Fewer or greater electrodes may be employed. For example, the counter and reference electrode functions may be served by a single counter/reference electrode, there may be more than one working electrode and/or reference electrode and/or counter electrode, etc.

The electronics of the on-skin sensor control unit and the sensor are operated using a power supply 207, e.g., a battery.

The analog interface 201 is coupled via the conductive contacts of the sensor control unit 102 to one or more sensors 101. The analog interface 201 in one embodiment is configured to receive signals from and to operate the sensor(s). For example, in one embodiment, the analyte interface 201 may obtain signals from sensor 101 (FIG. 1) using amperometric, coulometric, potentiometric, voltammetric, and/or other electrochemical techniques. For example, to obtain amperometric measurements, the analog interface 201 includes a potentiostat that provides a constant potential to a sensor. In other embodiments, the analog interface 201 includes an amperostat that supplies a constant current to a sensor and can be used to obtain coulometric or potentiometric measurements.

The signal from the sensor 101 (FIG. 1) generally has at least one characteristic, such as, for example, current, voltage, or frequency, or the like, which varies with the concentration of the analyte that the sensor 101 is monitoring. For example, if the analog interface 201 operates using amperometry, then the current signal from the sensor varies with variation in the monitored analyte concentration. Referring back to FIG. 2, one or more of the components of the data processing unit 102, including, for example, the processor 204, the analog interface 201, and/or the RF transmitter/receiver 206 may include circuitry which converts the information-carrying portion of the signal from one characteristic to another. For example, the one or more components of the data processing unit 102 such as the processor 204, the analyte interface 201, and/or the RF transmitter/receiver 206 may include a current-to-voltage or current-to-frequency converter. The purpose of this conversion may be to provide a signal that is, for example, more easily transmitted, readable by digital circuits, and/or less susceptible to noise contributions.

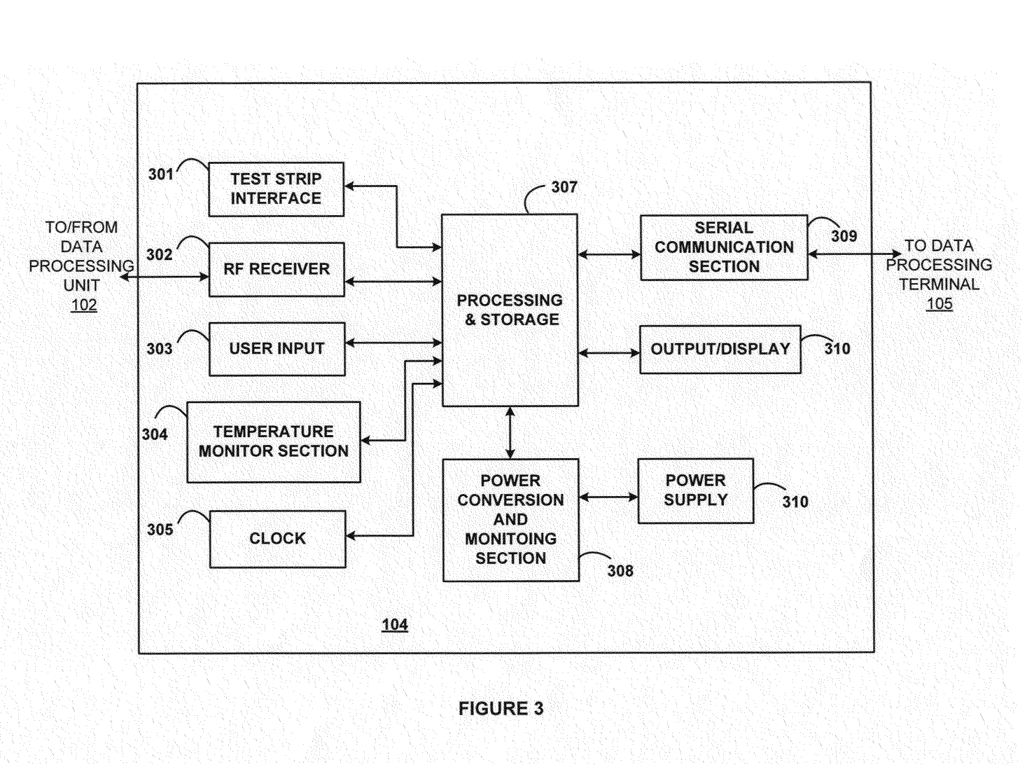

FIG. 3 is a block diagram of an embodiment of a receiver/monitor unit such as the primary receiver unit 104 of the data monitoring and management system shown in FIG. 1. The primary receiver unit 104 includes one or more of: a blood glucose test strip interface 301, an RF receiver 302, an input 303, a temperature detection section 304, and a clock 305, each of which is operatively coupled to a processing and storage section 307. The primary receiver unit 104 also includes a power supply 306 operatively coupled to a power conversion and monitoring section 308. Further, the power conversion and monitoring section 308 is also coupled to the receiver processor 307. Moreover, also shown are a receiver serial communication section 309, and an output 310, each operatively coupled to the processing and storage unit 307. The receiver unit 104 may include user input and/or interface components or may be free of user input and/or interface components.

In certain embodiments, the test strip interface 301 includes a glucose level testing portion to receive a blood (or other body fluid sample) glucose test or information related thereto. For example, the interface 301 may include a test strip port to receive a glucose test strip. The processing and storage unit 307 of the receiver unit 104 may in one embodiment determine the glucose level of the test strip, and optionally display (or otherwise notice) the glucose level on the output 310 of the primary receiver unit 104. Any suitable test strip may be employed, e.g., test strips that only require a very small amount (e.g., one microliter or less, e.g., 0.5 microliter or less, e.g., 0.1 microliter or less), of applied sample to the strip in order to obtain accurate glucose information, e.g. FreeStyle® and Precision® blood glucose test strips available from Abbott Diabetes Care Inc., of Alameda, Calif. Glucose information obtained by the in vitro glucose testing device may be used for a variety of purposes, computations, signal processing and/or data analysis. For example, the information may be used to calibrate sensor 101, confirm results of the sensor 101 to increase the confidence thereof (e.g., in instances in which information obtained by sensor 101 is employed in therapy related decisions), or used in conjunction with the determination of a medication dosage amount such as, for example, a bolus insulin dose, etc.

In further embodiments, the data processing unit 102 and/or the primary receiver unit 104 and/or the secondary receiver unit 105, and/or the data processing terminal/infusion section 105 may be configured to receive the blood glucose value wirelessly over a communication link from, for example, a blood glucose meter. In further embodiments, a user manipulating or using the analyte monitoring system 100 (FIG. 1) may manually input the blood glucose value using, for example, a user interface (for example, a keyboard, keypad, voice commands, and the like) incorporated in the one or more of the data processing unit 102, the primary receiver unit 104, secondary receiver unit 105, or the data processing terminal/infusion section 105.

Additional detailed descriptions are provided in U.S. Pat. Nos. 5,262,035; 5,264,104; 5,262,305; 5,320,715; 5,593,852; 6,175,752; 6,560,471; 6,746,582, 7,299,082 and in application Ser. No. 10/745,878 filed Dec. 26, 2003 entitled “Continuous Glucose Monitoring System and Methods of Use”, each of which is incorporated herein by reference.

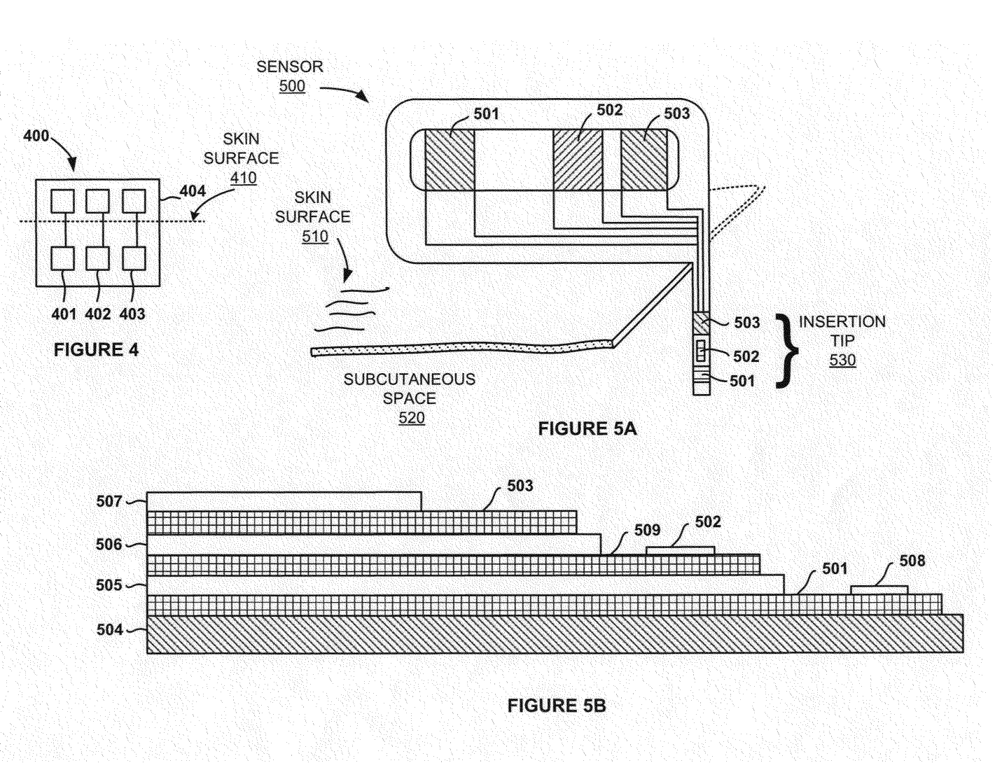

FIG. 4 schematically shows an embodiment of an analyte sensor in accordance with the present invention. This sensor 400 embodiment includes electrodes 401, 402 and 403 on a base 404. Alternatively, a sensor may be a wire-type sensor, e.g., as described in U.S. Pat. No. 6,284,478, the disclosure of which is herein incorporated by reference. For example, one or more wires may make up the sensor, e.g., with insulating material therebetween. In certain embodiments, a wire-type sensor includes a first wire, e.g., a working electrode, and a second wire, e.g., a counter electrode or reference electrode or counter/reference electrode, helically wound around at least a portion of the first wire, with an insulating material therebetween. Electrodes (and/or other features) may be applied or otherwise processed using any suitable technology, e.g., chemical vapor deposition (CVD), physical vapor deposition, sputtering, reactive sputtering, printing, coating, ablating (e.g., laser ablation), painting, dip coating, etching, and the like. Materials include but are not limited to aluminum, carbon (such as graphite), cobalt, copper, gallium, gold, indium, iridium, iron, lead, magnesium, mercury (as an amalgam), nickel, niobium, osmium, palladium, platinum, rhenium, rhodium, selenium, silicon (e.g., doped polycrystalline silicon), silver, tantalum, tin, titanium, tungsten, uranium, vanadium, zinc, zirconium, mixtures thereof, and alloys, oxides, or metallic compounds of these elements.

The sensor 400 may be wholly implantable in a user or may be configured so that only a portion is positioned within (internal) a user and another portion outside (external) a user. For example, the sensor 400 may include a portion positionable above a surface of the skin 410, and a portion positioned below the skin, i.e., trancutaneous. In such embodiments, the external portion may include contacts (connected to respective electrodes of the second portion by traces) to connect to another device also external to the user such as a control and/or transmitter unit. While the embodiment of FIG. 4 shows three electrodes side-by-side on the same surface of base 404, other configurations are contemplated, e.g., fewer or greater electrodes, some or all electrodes on different surfaces of the base or present on another base, some or all electrodes stacked together, electrodes of differing materials and dimensions, etc.

FIG. 5A shows a perspective view of an embodiment of an electrochemical analyte sensor 500 having a first portion (which in this embodiment may be characterized as a major portion) positionable above a surface of the skin 510, and a second portion (which in this embodiment may be characterized as a minor portion) that includes an insertion tip 530 positionable below the skin, e.g., penetrating through the skin and into, e.g., the subcutaneous space 520, in contact with the user's biofluid such as interstitial fluid. Contact portions of a working electrode 501, a reference electrode 502, and a counter electrode 503 are positioned on the portion of the sensor 500 situated above the skin surface 510. Working electrode 501, a reference electrode 502, and a counter electrode 503 are shown at the second section and particularly at the insertion tip 530. Traces may be provided from the electrode at the tip to the contact, as shown in FIG. 5A. It is to be understood that greater or fewer electrodes may be provided on a sensor. For example, a sensor may include more than one working electrode and/or the counter and reference electrodes may be a single counter/reference electrode, etc.

FIG. 5B shows a cross sectional view of a portion of the sensor 500 of FIG. 5A. The electrodes 510, 502 and 503, of the sensor 500 as well as the substrate and the dielectric layers are provided in a layered configuration or construction. For example, as shown in FIG. 5B, in one aspect, the sensor 500 (such as the sensor unit 101 FIG. 1), includes a substrate layer 504, and a first conducting layer 501 such as carbon, gold, etc., disposed on at least a portion of the substrate layer 504, and which may provide the working electrode. Also shown disposed on at least a portion of the first conducting layer 501 is a sensing layer 508.

A first insulation layer such as a first dielectric layer 505 is disposed or layered on at least a portion of the first conducting layer 501, and further, a second conducting layer 509 may be disposed or stacked on top of at least a portion of the first insulation layer (or dielectric layer) 505. As shown in FIG. 5B, the second conducting layer 509 may provide the reference electrode 502, and in one aspect, may include a second material such as conducting or semi-conducting material, e.g., a layer of silver/silver chloride (Ag/AgCl), gold, etc. If more than one material such as more than one conducting material is used for any of the electrodes, the different materials may have the same or different electrical conductivity. For example, different materials, if used, may have the same, greater, or less electrical conductivity with respect to each other.

A second insulation layer 506 such as a dielectric layer in one embodiment may be disposed or layered on at least a portion of the second conducting layer 509. Further, a third conducting layer 503 may provide the counter electrode 503. It may be disposed on at least a portion of the second insulation layer 506. Finally, a third insulation layer may be disposed or layered on at least a portion of the third conducting layer 503. In this manner, the sensor 500 may be layered such that at least a portion of each of the conducting layers is separated by a respective insulation layer (for example, a dielectric layer). The embodiment of FIGS. 5A and 5B show the layers having different lengths. Some or all of the layers may have the same or different lengths and/or widths.

In certain embodiments, some or all of the electrodes 501, 502, 503 may be provided on the same side of the substrate 504 in the layered construction as described above, or alternatively, may be provided in a co-planar manner such that two or more electrodes may be positioned on the same plane (e.g., side-by side (e.g., parallel) or angled relative to each other) on the substrate 504. For example, co-planar electrodes may include a suitable spacing there between and/or include dielectric material or insulation material disposed between the conducting layers/electrodes. Furthermore, in certain embodiments, one or more of the electrodes 501, 502, 503 may be disposed on opposing sides of the substrate 504. In such embodiments, contact pads may be one the same or different sides of the substrate. For example, an electrode may be on a first side and its respective contact may be on a second side, e.g., a trace connecting the electrode and the contact may traverse through the substrate.

As noted above, analyte sensors may include an analyte-responsive enzyme to provide a sensing component or sensing layer. Sensing layers, as well as optional additional layers or features (e.g., mass transport limiting layers and/or biocompatible layers, and/or interferent eliminating layers, etc.) are described, e.g., in U.S. Pat. Nos. 5,593,852; 6,881,551; 6,932,894, the disclosures of which are herein incorporated by reference.

The electrochemical sensors may employ any suitable measurement technique, e.g., may detect current, may employ potentiometry, etc. Techniques may include, but are not limited to amperometry, coulometry, voltammetry. In some embodiments, sensing systems may be optical, colorimetric, and the like. In certain embodiments, the sensing system detects hydrogen peroxide to infer glucose levels. For example, a hydrogen peroxide-detecting sensor may be constructed in which a sensing layer includes enzyme such as glucose oxides, glucose dehydrogensae, or the like, and is positioned proximate to the working electrode. The sensing layer may be covered by one or more layers, e.g., a membrane that is selectively permeable to glucose. Once the glucose passes through the membrane, it may be oxidized by the enzyme and reduced glucose oxidase can then be oxidized by reacting with molecular oxygen to produce hydrogen peroxide.

Embodiments of the subject invention also include sensors used in sensor-based drug delivery systems. The system may provide a drug to counteract the high or low level of the analyte in response to the signals from one or more sensors. Alternatively, the system may monitor the drug concentration to ensure that the drug remains within a desired therapeutic range. The drug delivery system may include one or more (e.g., two or more) sensors, a processing unit such as a transmitter, a receiver/display unit, and a drug administration system. In some cases, some or all components may be integrated in a single unit. A sensor-based drug delivery system may use data from the one or more sensors to provide necessary input for a control algorithm/mechanism to adjust the administration of drugs, e.g., automatically or semi-automatically. As an example, a glucose sensor may be used to control and adjust the administration of insulin from an external or implanted insulin pump.

Analyte sensors may be positioned in a user manually or using a device that automatically positions the sensor upon actuation. In certain embodiments, a sensor may be inserted using an insertion device. A kit may be provided that includes some or all analyte sensing components, e.g., an analyte sensor may be preloaded or loadable within an inserter, where the inserter may be provided to the user in a “cocked” or “un-cocked”, state. After preparing an insertion site on the skin, the patient may remove a protective liner from an adhesive on-body unit to expose adhesive located beneath the on-body unit. The on-body unit, with or without the inserter attached, may then be applied to the patient's skin at the insertion site. An actuator may be contacted to cause the inserter to fire, thereby inserting the sensor into the patient's skin, in many embodiments through a port or exposed area of an on-body unit. Once the sensor has been inserted into the skin, the patient may remove the inserter from the on-body unit if configured to be removed, to remove the inserter away from the on-body unit. Analyte sensor insertion and insertion devices are described, e.g., in U.S. Pat. Nos. 6,284,478; 6,175,752; 7,381,184; and in U.S. patent application Ser. No. 11/617,698 entitled “Medical Device Insertion”, in U.S. patent application Ser. No. 11/535,983 entitled “Method and Apparatus for Providing Analyte Sensor Insertion”, U.S. patent application Ser. No. 11/192,773 entitled “Inserter and Methods of Use”, in U.S. patent application Ser. No. 11/530,472 entitled “Method and System for Providing Integrated Analyte Sensor Insertion Device and Data Processing Unit” the disclosures of each of which are herein incorporated by reference for all purposes.

As described, embodiments include analyte systems that include a transcutaneous analyte sensor and an on-body unit wherein the depth of sensor insertion is selectable, i.e., pre-determined, to a selected depth from amongst a plurality of possible depths, before the sensor is inserted at the target site of a user. Analyte sensors may be configured to be positioned in a variety of locations, e.g., the dermis, subcutaneous adipose tissue, muscle, etc., and embodiments are provided that enable sensor depth selection so that a particular insertion depth may be selected, e.g., from a plurality of predetermined insertion depths, and a sensor may then be positioned at the selected depth. A variety of variable depth insertion systems are described herein, and with particular respect to variable depth features, as noted above features of each may be employed independently of any of the others, or features of one or more embodiments may be combined. Additional description is provided in pending U.S. patent application Ser. No. 12/416,126 entitled “Shallow Implantable Analyte Sensor with Rapid Physiological Response” assigned to assignee of the present application, the disclosure of which is incorporated by reference for all purposes.

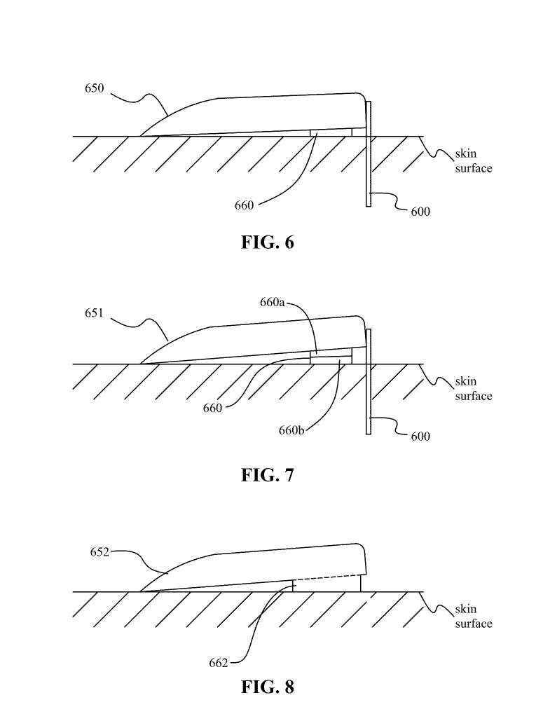

In certain embodiments, an on-body unit includes an adjustable and variable depth controller to determine the effective sensor insertion depth beneath the skin of a user. For example, FIG. 6 shows such an embodiment and includes sensor 600, shown transcutaneously positioned and mated with on-body unit 650 which as described herein may be a sensor control unit and/or mounting unit and/or may be a mounting unit for a sensor control unit, or the like. In one aspect, the on-body unit 650 is mateable with sensor 600 when the sensor is positioned in a user for analyte monitoring. In many embodiments, though the depth to which the sensor 600 is positioned is adjustable, the distance between the sensor 600 and the on-body unit 650 is fixed. In other words, the distance between the sensor 600 and on-body unit 650 remains constant, but the distance between the on-body unit 650 and the skin may be varied.

In certain embodiments, on-body unit 650 includes a sensor port or other sensor-accessible area through or about which sensor 600 enters the user. Sensor 600 may be inserted into, e.g., the subcutaneous tissue of a user, through the sensor port or other area of an on-body unit 650. The on-body unit 650 may be placed on the skin with a sensor 600 being positioned through the sensor port, or before a sensor 600 is aligned therewith for insertion, and the sensor 600 positioned thereafter. If the housing of an on-body unit 650 has, for example, a base and a cover, then the cover may be removed to allow a user to guide a sensor into the proper position, e.g., for contact with conductive contacts of the on-body unit 650 if present or other mateable unit. Alternatively, if conductive contacts are within the on-body unit 650, a user may slide a sensor 600 into the housing of the on-body unit 650 until contact is made between the electrical contacts of the sensor 600 and the electrical contacts of the on-body unit 650 if present or other mateable unit. In some embodiments, the conductive contacts may be located on the exterior of the housing of an on-body unit 650. In these embodiments, a user may guide the electric contacts of the sensor 600 into contact with the conductive contacts of the on-body unit 650 or other mateable unit. In some cases, a guiding structure may be provided to guide a sensor into the proper position.

As shown in the embodiment of FIG. 6, depth controller 660 is in the form of an offset, e.g., one or more spacers, provided between the skin surface, and thus at least a portion of sensor 600 that is positioned beneath the skin surface, and the on-body unit 650. In one aspect, as shown in FIG. 6, the depth controller 660 is sandwiched between the skin-facing surface of the on-body unit 650 and the skin surface.

More than one offset may be employed at any given time to increase/decrease the distance between the on-body unit and the skin surface, effectively determining the depth of the sensor 600 beneath the skin surface. In the embodiments in which a plurality of offsets is provided, all or some may be the same or different sizes, e.g., thicknesses and/or lengths, and/or widths and/or diameters. The offsets(s) may be any suitable shape, size and thickness, where shapes that conform to the shape of a body part may be used. For example, in one embodiment, the offset thickness may have a variable dimension, e.g., a variable width and/or length and/or thickness. An offset may be wedge shaped, e.g., may be thicker at one end and taper down to be thinner at the other end to control sensor depth. In this manner, a tight seal may be made between the on-body unit 650 and the skin of a user to eliminate moisture, dirt, etc. from entering the area. The wedged thickness offset may be substantially the same size and/or shape as an on-body unit 650 or may extend beyond part or all of the perimeter of the on-body unit 650 or may be smaller in at least one dimension. In certain embodiments, the offset may be substantially the same shape and/or size as the on-body unit 650.

FIG. 7 shows on-body unit 651 with offset 660 in the form of a plurality of offset members or spacers 660a and 660b positioned to provide greater distance between the skin surface, and thus at least a portion of sensor 600 that is positioned beneath the skin surface, and the on-body unit 651, to provide a more shallow depth of sensor positioning than the single offset of the embodiment of FIG. 6, if the single offset 660 of FIG. 6 were to have a thickness less than that of the plurality of offsets 660a, 660b of FIG. 7.

Offsets may be integrated with an on-body unit and therefore may be configured to be selectively moveable into position to affect a selected depth of sensor insertion, or may be separate from on-body unit for appropriate placement. For example, one or more offsets may be integrated with the on-body unit (e.g., in or on the housing of the on-body unit) and configured to extend from the on-body unit a selective distance. FIG. 8 shows an embodiment that includes an on-body unit 652 that includes a depth controller 662 in the form of an integrated offset that can extend (e.g., telescope or otherwise be moved) from the on-body unit 652, and optionally retract back into the on-body unit 652. Accordingly, the depth controller 662 may be selectively moveable from a first position substantially within or on the on-body unit 652 to a second position in which at least a portion of the depth controller 662 extends from the on-body unit 652 a selective distance. A depth controller 662 may also be selectively retractable to a position within or on the on-body unit 652.

A depth controller may be any suitable configuration and material, including, but not limited to, polymers, elastomers, metals, alloys, adhesives, and the like. For example, in certain embodiments the on-body unit is attachable to the skin with an adhesive. An offset may include one or more additional adhesive layers and/or and increased thickness of the on-body adhesive layer. Plurality of adhesive layers may be provided which may be used to define the sensor insertion depth.

In certain embodiments, e.g., for a wire-type sensor (see for example U.S. Pat. No. 6,284,478, the disclosure of which is herein incorporated by reference), a sensor inserter (such as one that includes an introducer needle) includes an actuator such as a knob or dial the like. Actuating the actuator (e.g., turning a knob) controls the skin penetration depth of the sensor. For example, turning a knob correspondingly has two effects: it coils the wire-type sensor to reduce its inserted length, and alters the penetration depth of the introducer needle (e.g., external or internal spacer foot could be raised/lowered setting max penetration depth) to match the sensor length. The angle of a wire-type sensor insertion may be controlled by the same (or different) knob and setting the depth may automatically adjust the angle of insertion (e.g., about 5.5 mm at about 90 degrees and about 7.8 mm at about 45 degrees.

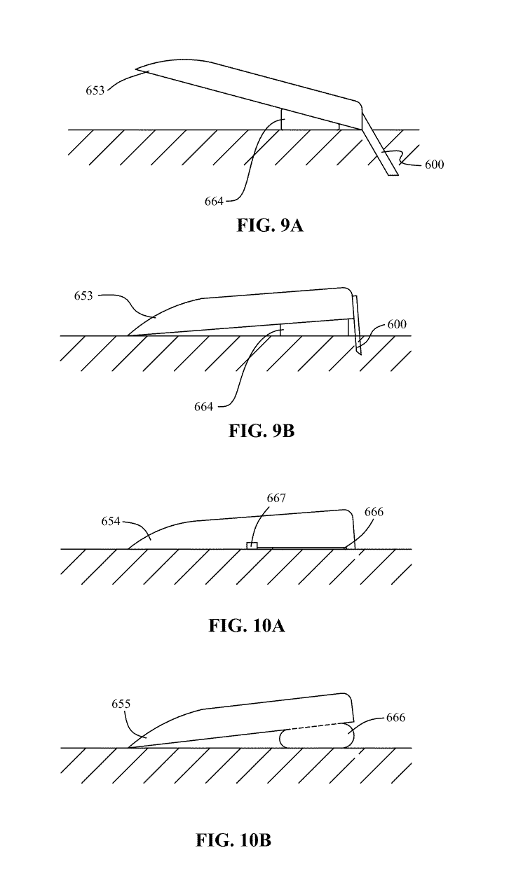

In certain embodiments, sensor insertion depth may be determined at least by selectively controlling the angle of sensor insertion. Embodiments include depth controllers that selectively adjust the angle that the sensor is positioned relative to the skin. Angles of insertion may range from about 1 to about 5 degrees to about 85 to about 90 degrees, with respect to the plane of skin surface. FIGS. 9A and 9B show embodiments that include on-body unit 653 and a depth controller 664 that is configured to control the sensor insertion angle. FIG. 9A shows an embodiment in which the sensor 600 is angled relative to the on-body unit 653, and FIG. 9B shows an embodiment in which the sensor 600 is approximately perpendicular to the on-body unit 653 and the whole assembly is angled relative to the skin surface to accomplish depth adjustment of the sensor 600.

FIGS. 10A and 10B show an embodiment that includes on-body unit 654 and a depth controller in the form of an adjustable bladder 666. In the embodiment of FIGS. 10A and 10B, the bladder 666 is integrated with the on-body unit 654 and is configured to extend a selectable distance from the on-body unit 654 to affect a selected sensor depth. In certain embodiments, the bladder is integrated with a mounting unit housing and/or a sensor control unit housing. In certain embodiments a bladder may be separate from an on-body unit and mateable therewith. FIG. 10A shows bladder 666 in a deflated state within a cavity of on-body unit 654. Bladder 666 is connected to a filling source 667 (gas, liquid, or solid (e.g., gel)). Upon actuation, an amount of filling material required to insert the sensor a selected depth fills bladder 666 to expand it and increase the distance between the on-body unit 655 and the skin, as shown in FIG. 10B.

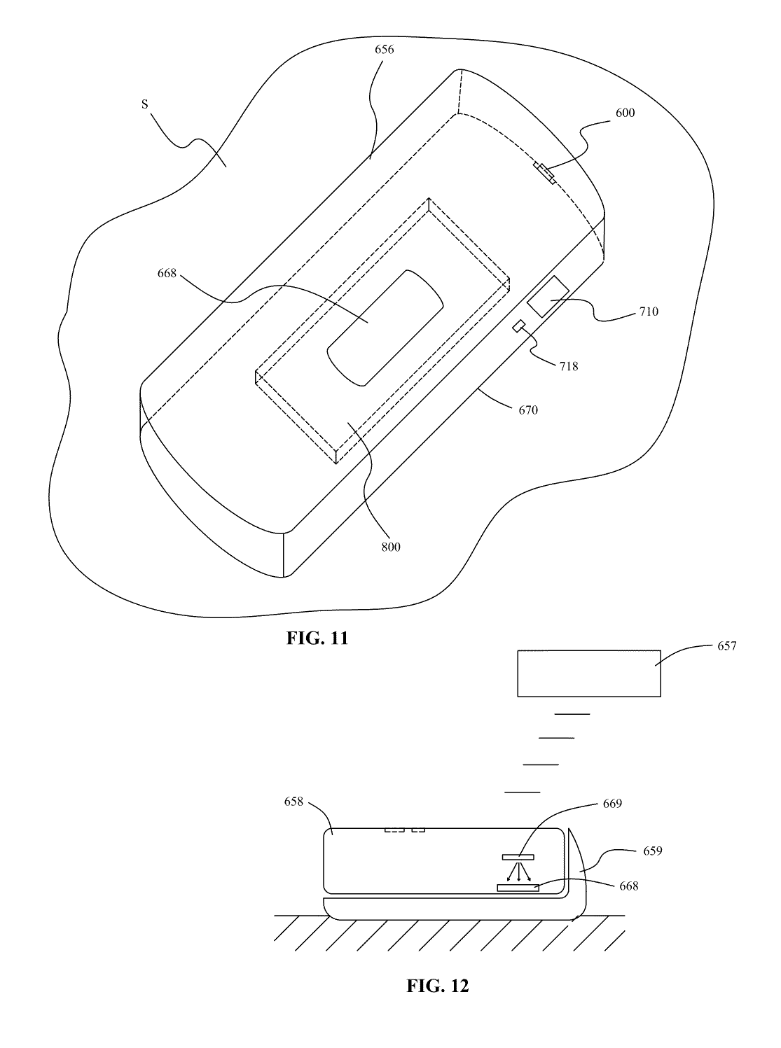

FIG. 11 shows an embodiment of an on-body mounting unit 656 that is mateable with a sensor control unit (not shown), one or both of which may include electrical contacts for contacting corresponding electrical contacts of an analyte sensor when at least a portion of the sensor is positioned beneath skin of a user. Mounting unit 656 is shown adhered to a patient's skin S with sensor 600 shown inserted and adjacent the mounting unit 656. Mounting unit 656 includes a body-attachment member 670, in this embodiment shown as an adhesive member on a surface thereof. A senor control unit may be mated with mounting unit 656, as shown e.g., in FIG. 12, e.g., slid into place or otherwise connected with features on one or both, e.g., by use of grooves, latches, interlocking structures, detachable or permanent adhesives and the like. As described herein, a sensor control unit may include an RF transmitter, RF receiver or RF transceiver, e.g., for one and/or two-way communication with a secondary device, however other modes of communication may be employed e.g., infrared (IR), etc. The circuitry of a sensor control unit makes electrical contact with the electrical contacts of sensor 600 after the sensor control unit is mated with mount 656 which registers the conductive contacts of the sensor with the conductive contacts of the sensor control unit. Once initialization and synchronization procedures are completed, electrochemical measurements from sensor 600 may be sent, e.g., wirelessly, from the sensor control unit to the secondary device such as a portable receiver.

As shown in FIG. 11, mounting unit 656 includes an optional depth control holder 800 to hold a selective depth control member 668 that is integrated with mounting unit 656, e.g., in a recess, cavity, e.g., an interior space, for deployment from the mounting unit to a selected depth at a selected sensor insertion time. Depth controller 668 is moved from a first position where it is substantially within mounting unit 656 to a second or depth adjust position by a mover, which is configured to move depth control member 668 a selected distance according to a desired sensor depth. The mover may take any suitable form, including but not limited to a spring, ratchet/pawl system, screw, plunger, jack, tensioner, or the like.

Also included may be an optional depth selector 710, e.g., a button, latch, knob, thumbwheel, etc. coupled to the depth controller so that actuation of the depth selector corresponds to actuation of the depth controller to selected depth. As used herein, the term “depth selector” is non limiting and includes rotating dials, push buttons, tactile actuators or other elements, translating slide mechanisms, or other movable stop mechanisms, that are manipulable in any way by the user. A depth selector 710 may further include an indexer (not shown) to allow the user to increment the depth ranges, and accordingly the penetration depth, through a plurality of discrete positions. Also included may be an optional depth indicator 718 to indicate, e.g., with indicia, the set depth of penetration of a sensor into the skin.

FIG. 12 shows an embodiment of an-body mounting unit 659 mated with a sensor control unit 658 that includes a selective depth control member integrated therewith. Mover 669 is position to act on depth control member 668 to move it a selected distance according to a desired sensor depth. For example, mover 669 may move the sensor control unit, around or through mounting unit 659 (e.g., through a clearance) to adjust the distance of the sensor control unit and/or mounting unit relative to the skin, which correspondingly adjusts the distance of a sensor for depth adjustment.

In certain embodiments, sensors of different lengths and/or pre-set angles may be provided so that a particular sensor may be selected from a plurality to achieve a desired insertion depth. For example, sensors having lengths ranging from about 1 mm to about 20 mm may be provided. Embodiments include sensor lengths of about 1 mm to about 20 mm and positionable at angles of about 1 to about 90 degrees relative to the skin, e.g., angles of about 1 to about 45 degrees relative to the skin. In certain embodiments, a sensor may be bendable to a particular angle depending on a desired depth.

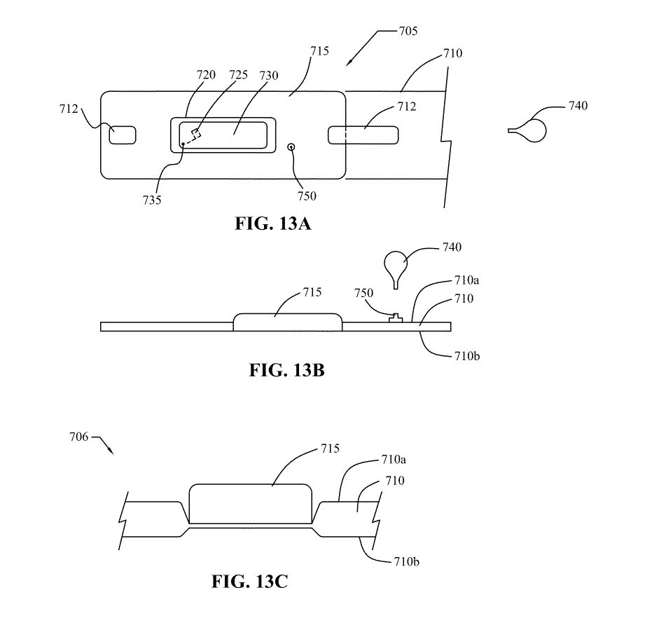

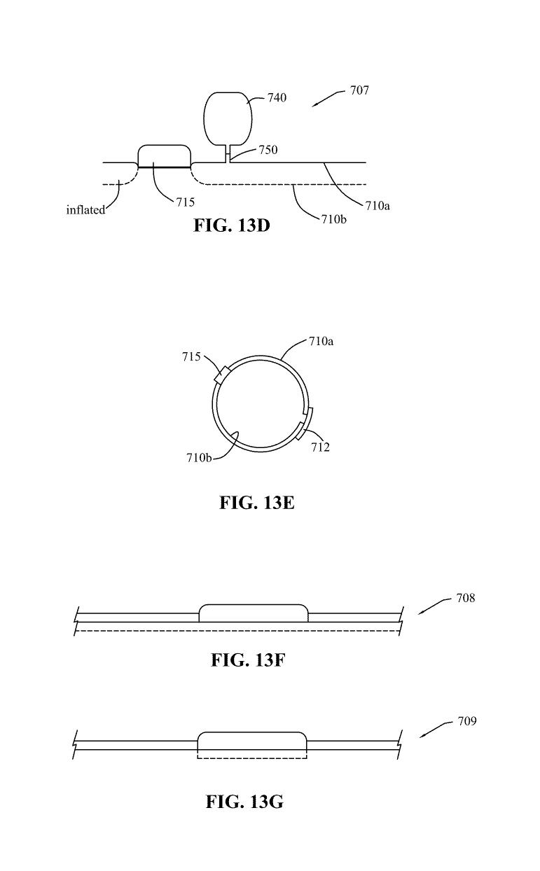

FIGS. 13A-13E show embodiments of on-body assemblies that include an inflatable cuff. In certain embodiments the inflatable cuff may be configured to selectively adjust the depth of insertion of a sensor. Accordingly, embodiments include sensor depth controllers in the form of inflatable cuffs. Such a cuff may be purchased ready for application and fully assembled, or may require partial assembly by the user. Additional description is provided in pending U.S. patent application Ser. No. 11/240,257 entitled “Integrated Transmitter Unit and Sensor Introducer Mechanism and Methods of Use” assigned to assignee of the present application, the disclosure of which is incorporated by reference for all purposes.

As shown in FIGS. 13A-13E, the assemblies include a band (cuff) 710 with a mounting unit 715, which mounting unit area may or may not inflate. Mounting unit 715 may include an optional mateable cartridge 720 containing a sensor 725 and a sensor inserter 730. Cartridge 720 and/or sensor 725 and/or inserter 730 may be provided pre-assembled, e.g., integrated with, band 710, or may be provided for user attachment. For example, in certain embodiments, an inserter cartridge is provided to a user pre-mounted on the inflatable cuff with a sensor positioned therewith for insertion.

All or only a portion of an inflatable assembly may be configured to inflate, where whether a portion is inflatable or not may be fixed, e.g., during manufacture, or may be selectable, e.g., by a user. FIGS. 13A and 13B show an embodiment of an inflatable assembly 705 in a first or deflated state. A cuff 710 may include a first side 710a and a second side 710b and one or both sides of a cuff 710 may be inflatable, e.g., selectively. FIG. 13C shows cuff assembly embodiment 706 in a second or inflated state in which both sides 710a and 710b are inflated. FIGS. 13D and 13E show cuff assembly embodiment 707 in a second or inflated state in which only one side, in this particular embodiment side 710b, is inflated. FIG. 13F shows inflation assembly embodiment 708 that is configured to inflate along its entirety (inflation indicated with dashed lines), and FIG. 13G shows cuff assembly embodiment 709 that is configured to inflate in the mount area, e.g., exclusively or in addition to other portions of the assembly.

A cuff may be constructed of any suitable material. In certain embodiments, a cuff may include one or more polymer layers. If a plurality of polymer layers is employed, any of the layers may be of the same or different thickness and/or flexibility of any of the other films. Layers may be sealed together (e.g., welded) to provide a unitary piece. For example, the layers may be heat sealed (inductive, RF, pulsation, etc.). The construction of the material(s) may be selected to limit inflation beyond a predetermined amount, e.g., limit it so that the greatest inflation still provides for a sensor to be inserted under the skin of a user. The properties of the material(s) may allow for a certain amount of breath-ability for the skin. The surface that is opposite the skin may be puncture resistant and all materials may be water proof and environmentally resistant.

The cuff may be in the form of an open band, which is configured to be closeable with a closure to form a closed band, e.g., by a user, or may be provided as a closed band, e.g., a continuous unit. In any event, the cuff may be positioned about the body of a user, i.e., arm, leg, abdomen, etc, and held securely in place by any suitable closure mechanism 712 (see e.g., FIG. 13A), including but not limited to, Velcro, adhesive tape, hook and loop, buttons, etc.

Referring back to FIG. 13A, in certain embodiments, a sensor enters the skin of a user through a sensor hole 735, which is located on the cuff 710, e.g., within the mount area. The cuff 710 is configured to inflate a selectable amount to affect a selected sensor depth. The cuff 710 may be partially or fully inflatable. FIG. 13B shows cuff 710 in a deflated state. Inflation may be accomplished by any suitable mechanism which may be integral with the cuff or a separate component. For example, inflation may be accomplished by use of a fluid, air bulb or hand pump 740 that inflates the cuff through an inflation valve 750. A low pressure, low profile check valve may also be included to prevent deflation when inflation ceases, e.g., when an inflation device such as an air bulb or hand pump is removed in those embodiments in which an inflation device is attachably removable from the cuff. Upon actuation, fluid fills cuff 710 to expand it and increase the distance between at least mount unit 715 of the on-body unit and the skin as shown in FIG. 13C.

For example, after the entire assembly may be positioned at a sensor insertion site and is secured, the user may then attach, if not already attached, an inflation device to inflate the cuff to a desired level depending on a particular sensor insertion depth desired. The sensor insertion may be done manually or automatically. The sensor may be inserted into the skin of the user through the sensor hole of the cuff, e.g., using an introducer cartridge. In certain embodiments a sensor may be inserted by a force applied to an introducer cartridge mounted on the cuff assembly, e.g., by a user's finger. After insertion, the introducer may be removed from the skin, leaving the sensor at the selected depth, and in correct position for electrically connecting to a sensor control unit, or the introducer may be retracted into the cuff.

Once the sensor is in place at the desired depth, the cartridge may be removed from the mount if so configured for removability. A sensor control unit may then be mounted in position on the mount affixed to the cuff, to establish an electrical connection between the sensor and the control unit. A compressible seal may be provided about the electrical contacts of the sensor and/or control unit to prevent moisture and dirt from damaging the integrity of the electrical connection.

Accordingly, as described herein, embodiments include in vivo glucose monitoring systems that may include: an in vivo glucose sensor comprising at least a portion that is positionable beneath skin to contact biological fluid for a period of time of glucose monitoring, a working electrode and at least one additional electrode, at least one electrical contact coupled to the electrodes, and glucose-responsive enzyme; and an on-body unit comprising a sensor contacting portion to contact the in vivo sensor when at least a portion of the sensor is positioned beneath skin, and comprising a selective depth controller to selectively adjust the depth of the sensor beneath the skin without adjusting the distance between the sensor and the on-body unit so that the distance between the sensor and on-body unit is fixed and the sensor extends the same distance from the on-body unit regardless of the selected depth.

An on-body unit may include a mounting unit, or a sensor control unit or a sensor control unit mountable in a mounting unit.

A depth controller may includes an offset, and an offset may include one or more spacers. For example a system may include at least two spacers, and the at least two spacers may be the same size or may be different sizes. A spacer may have a uniform dimension or a variable dimension, e.g., the dimension may be a thickness.

A depth controller may be integrated with the on-body unit.

A depth controller may be selectively moveable from a first position substantially within or on the on-body unit to a second position in which at least a portion of the depth controller extends from the on-body unit a selective distance.

A depth controller may be retractable to a position within or on the on-body unit.

A depth controller may be separate from the on-body unit.

A depth controller may selectively adjust the angle that the sensor is positioned relative to skin, e.g., angle ranges from about 1 to about 90 degrees.

A depth controller may be an adjustable bladder.

An on-body unit may include a user-actuatable depth selector coupled to the depth controller. the depth selector comprises an indexer that increments the depth ranges through a plurality of discrete positions.

An on-body unit may include an inflatable cuff.

A sensor may be a planar sensor and/or a wire-type sensor.

Embodiments include methods of inserting a glucose sensor a selected in vivo tissue insertion depth to monitor glucose while the sensor is at the selected depth and at least a portion of the sensor is mated to an on-body unit. The methods may include: selecting an in vivo sensor insertion depth relative to skin from a plurality of selectable depths to insert at least a portion of the glucose sensor under skin to the selected depth; adjusting a selective depth controller to correspond to the selected in vivo sensor insertion depth; and inserting the glucose sensor to the selected depth with the depth controller, wherein the distance between the sensor and the on-body unit is the same regardless of the depth to which the sensor is inserted.

Adjusting the selective depth controller may include modulating the distance between the skin and the mounting unit to correspondingly adjust the depth.

An on-body unit employed in the methods may include a mounting unit, or a sensor control unit or a sensor control unit mounted in a mounting unit.

Methods may include contacting conductive contacts of the sensor with conductive contacts of the on-body. The conductive contacts of the on-body unit may be in or on a mounting unit. Conductive contacts of the on-body unit may be in or on a sensor control unit.

Methods may include employing a depth controller comprises one or more offsets.

Depth controllers employed in the methods may be integrated with the on-body unit.

Depth controllers employed in the methods may be selectively moveable from a first position substantially within or on the on-body unit to a second position in which at least a portion of the depth controller extends from the on-body unit a selective distance.

Depth controllers employed in the methods may be separate from the on-body unit.

Methods may include selecting an angle that the sensor is positioned relative to skin from a plurality of angles depending on a desired sensor depth, and inserting the sensor at the selected angle to position the sensor at the desired sensor depth.

An on-body unit employed in the methods may include an inflatable cuff.

Sensors employed in the methods may be planar sensors and/or wire-type sensors.

It is evident from the above that the above-described invention provides medical device insertion devices and methods, e.g., analyte insertion devices and methods such as analyte sensors that are configured for transcutaneous positioning. The above-described invention provides a number of advantages some of which are described above and which include, but are not limited to, ease of use, and user customization. Furthermore, the subject invention provides a patient with a high degree of confidence that the medical device is securely maintained in position on a body part. As such, the subject invention represents a significant contribution to the art.

While the present invention has been described with reference to the specific embodiments thereof, it should be understood by those skilled in the art that various changes may be made and equivalents may be substituted without departing from the true spirit and scope of the invention. In addition, many modifications may be made to adapt a particular situation, material, composition of matter, process, process step or steps, to the objective, spirit and scope of the present invention. All such modifications are intended to be within the scope of the claims appended hereto.

What is claimed is:

1. An in vivo glucose monitoring system, comprising:
an in vivo glucose sensor comprising at least a portion that is positionable beneath a skin surface to contact biological fluid for a period of time of glucose monitoring, a working electrode and at least one additional electrode, at least one electrical contact coupled to the electrodes, and a glucose-responsive enzyme; and
an on-body unit comprising a sensor contacting portion to contact the in vivo sensor when at least a portion of the sensor is positioned beneath skin, and comprising a selective depth controller to selectively adjust the depth of the sensor beneath the skin surface without adjusting the distance between the sensor and the on-body unit so that the distance between the sensor and on-body unit is fixed and the sensor extends the same distance from the on-body unit regardless of the selected depth.

2. The system of claim 1, wherein the on-body unit comprises a mounting unit, or a sensor control unit or a sensor control unit mountable in a mounting unit.

3. The system of claim 2, wherein the depth controller comprises an offset.

4. The system of claim 3, wherein the offset comprises one or more spacers.

5. The system of claim 4, wherein the system comprises at least two spacers, and the at least two spacers are the same size.

6. The system of claim 4, wherein the system comprises at least two spacers, and the at least two spacers are different sizes.

7. The system of claim 4, wherein a spacer has a variable dimension.

8. The system of claim 7, wherein the dimension is a thickness.

9. The system of claim 2, wherein the depth controller is integrated with the on-body unit.

10. The system of claim 9, wherein the depth controller is selectively moveable from a first position substantially within or on the on-body unit to a second position in which at least a portion of the depth controller extends from the on-body unit a selective distance.

11. The system of claim 10, wherein the depth controller is retractable to a position within or on the on-body unit.

12. The system of claim 2, wherein the depth controller is separate from the on-body unit.

13. The system of claim 2, wherein the depth controller selectively adjusts the angle that the sensor is positioned relative to skin.

14. The system of claim 13, wherein the angle ranges from about 1 to about 90 degrees.

15. The system of claim 2, wherein the depth controller is an adjustable bladder.

16. The system of claim 2, wherein the on-body unit comprises a user-actuatable depth selector coupled to the depth controller.

17. The system of claim 2, wherein the depth selector comprises an indexer that increments the depth ranges through a plurality of discrete positions.

18. The system of claim 2, wherein the on-body unit comprises an inflatable cuff

19. The system of claim 1, wherein the sensor is a planar sensor.

20. The system of claim 1, wherein the sensor is a wire-type sensor.

21. A method, comprising:
selecting an in vivo sensor insertion depth relative to skin from a plurality of selectable depths to insert at least a portion of the sensor under a skin surface to the selected depth;
adjusting a selective depth controller to correspond to the selected in vivo sensor insertion depth; and
inserting the sensor to the selected depth with the depth controller,
wherein the distance between the sensor and an on-body unit is the same regardless of the depth to which the sensor is inserted.

22. The method of claim 21, wherein the on-body unit comprises a mounting unit, or a sensor control unit or a sensor control unit mounted in a mounting unit.

23. The method of claim 22, wherein adjusting the selective depth controller comprises modulating the distance between the skin surface and the mounting unit to correspondingly adjust the depth.

24. The method of claim 21, comprising contacting one or more conductive contacts of the sensor with one or more conductive contacts of the on-body unit.

25. The method of claim 24, wherein the one or more conductive contacts of the on-body unit are in or on a mounting unit.

26. The method of claim 24, wherein the one or more conductive contacts of the on-body unit are in or on a sensor control unit.

27. The method of claim 24, wherein the depth controller comprises one or more offsets.

28. The method of claim 24, wherein the depth controller is integrated with the on-body unit.

29. The method of claim 28, wherein the depth controller is selectively moveable from a first position substantially within or on the on-body unit to a second position in which at least a portion of the depth controller extends from the on-body unit a selective distance.

30. The method of claim 24, wherein the depth controller is separate from the on-body unit.

31. The method of claim 24, including selecting an angle that the sensor is positioned relative to skin from a plurality of angles depending on a desired sensor depth, and inserting the sensor at the selected angle to position the sensor at the desired sensor depth.

32. The method of claim 24, wherein the on-body unit comprises an inflatable cuff.
